(19) United States
(12) Patent Application Publication
(10) Pub. No.: US 2024/0009653 A1
(43) Pub. Date: Jan. 11, 2024

(54) Catalyst, Method for Producing Catalyst, and Method for Producing alpha,beta-Unsaturated Aldehydes, alpha,beta-Unsaturated Carboxylic Acids and alpha,beta-Unsaturated Carboxylic Acid Esters

(71) Applicant: Mitsubishi Chemical Corporation, Tokyo (JP)

(72) Inventors: Kensuke Nishiki, Tokyo (JP); Yu Kurihara, Tokyo (JP); Takuro Watanabe, Tokyo (JP)

(73) Assignee: Mitsubishi Chemical Corporation, Tokyo (JP)

(21) Appl. No.: 18/371,234

(22) Filed: Sep. 21, 2023

Related U.S. Application Data
(63) Continuation of application No. PCT/JP2022/013473, filed on Mar. 23, 2022.

(30) Foreign Application Priority Data
Mar. 24, 2021 (JP) 2021-049748

(51) Int. Cl. B01J 23/887; B01J 23/00; B01J 37/04; B01J 37/08; B01J 6/00

(52) U.S. Cl. CPC B01J 23/8876; B01J 23/002; B01J 37/04; B01J 37/08; B01J 6/001

(57) ABSTRACT

An object of the present invention is to provide a catalyst with high selectivity for an α,β-unsaturated aldehyde, an α,β-unsaturated carboxylic acid, and the like. Problems are solved by a catalyst containing at least molybdenum and bismuth and having a B/A of 1.3 to 5 when a ratio of the amount of bismuth atoms to the amount of molybdenum atoms, calculated from ICP (inductively coupled plasma) atomic emission spectrometry is A, and a ratio of a peak area of bismuth atoms to a peak area of molybdenum atoms, measured by X-ray photoelectron spectrometry is B.

### CROSS-REFERENCE TO RELATED APPLICATION

This is a continuation of International Application PCT/JP2022/013473, filed on Mar. 23, 2022, and designated the U.S., and claims priority from Japanese Patent Application 2021-049748 which was filed on Mar. 24, 2021, the entire contents of which are incorporated herein by reference.

### TECHNICAL FIELD

The present invention relates to a catalyst, a method of producing the catalyst, and a method of producing an α,β-unsaturated aldehyde, an α,β-unsaturated carboxylic acid, and an α,β-unsaturated carboxylic acid ester.

### BACKGROUND ART

Methods of producing α,β-unsaturated aldehydes, α,β-unsaturated carboxylic acids, and the like by gas phase oxidation reactions in the presence of metal oxide catalysts using organic compounds such as propylene, isobutylene, t-butyl alcohol, methyl-t-butyl ether, and the like, have been known.

For example, Patent Document 1 describes a method of producing a mixed oxide catalyst containing at least molybdenum, bismuth, cobalt and/or nickel, and iron as a method of producing a catalyst used upon producing corresponding unsaturated aldehyde and unsaturated carboxylic acid from olefins.

Moreover, Patent Document 2 describes an example that a catalyst for synthesis of unsaturated aldehydes and unsaturated carboxylic acids with excellent catalytic activity and selectivity can be provided whereby the catalyst is composed of mixed oxide particles containing at least molybdenum, iron, and cobalt, and atomic ratios in a bulk composition and a surface composition, of the particles, satisfy specific conditions.

### RELATED ART DOCUMENTS

### Patent Documents

[Patent Document 1] Japanese Unexamined Patent Application Publication No. 2005-169311

[Patent Document 2] Japanese Unexamined Patent Application Publication No. 2011-115681

### SUMMARY OF THE INVENTION

### Problem to be Solved by the Invention

However, the present inventors have found that the catalysts described in Patent Documents 1 and 2 do not always have sufficient performance, and many byproducts may have been generated. Since these problems affect selectivity of α,β-unsaturated aldehydes, α,β-unsaturated carboxylic acids, and the like, further improvements in catalyst performance are actually desired. Therefore, catalyst physical properties have been required to be controlled from the viewpoint of further improvement of catalyst performance.

The present invention has been made in view of the aforementioned circumstances, and an object of the present invention is to provide a catalyst with high selectivity of target products such as an α,β-unsaturated aldehyde and an α,β-unsaturated carboxylic acid.

### Means for Solving the Problems

The present inventors have conducted diligent investigations in order to achieve the aforementioned object. As a result, the present inventors have found that in a catalyst containing at least molybdenum and bismuth, target products can be produced with high selectivity by adjusting a bismuth composition on the catalyst surface with respect to the entire catalyst.

Namely, the following is included in the present invention.

[1]: A catalyst comprising at least molybdenum and bismuth,
wherein when a ratio of the amount of bismuth atoms to the amount of molybdenum atoms, calculated from ICP (inductively coupled plasma) atomic emission spectrometry is A, and a ratio of a peak area of bismuth atoms to a peak area of molybdenum atoms, measured by X-ray photoelectron spectroscopy is B, a B/A is 1.3 to 5.
   [2]: The catalyst according to [1], wherein the B/A value is 1.5 to 4.
   [3]: The catalyst according to [1] or [2], wherein the B/A value is 1.7 to 3.
   [4]: The catalyst according to any one of [1] to [3], wherein the A value is 0.02 to 0.1.
   [5]: The catalyst according to any one of [1] to [4] , wherein the B value is 0.04 to 0.2.
   [6]: The catalyst according to any one of [1] to [5] , wherein the B value is 0.07 to 0.16.
   [7]: The catalyst according to any one of [1] to [6] , for use in production of an α,β-unsaturated aldehyde and/or an α,β-unsaturated carboxylic acid from an alkene, an alcohol, or an ether.
   [8]: The catalyst according to any one of [1] to [7] , wherein a catalyst composition is represented by the following formula (1):

MoₐBi_{b}Fe_{c}M_{d}XₑY_{f}Si_{g}Oₕ (1)

(wherein in the above formula (1), Mo, Bi, Fe, Si and O each represent molybdenum, bismuth, iron, silicon, and oxygen, respectively; M represents at least one element selected from the group consisting of cobalt and nickel; X represents at least one element selected from the group consisting of zinc, chromium, lead, manganese, calcium, magnesium, niobium, silver, barium, tin, tantalum, tungsten, antimony, phosphorus, boron, sulfur, selenium, tellurium, cerium and titanium; Y represents at least one element selected from the group consisting of cesium, lithium, sodium, potassium, rubidium, and thallium; a, b, c, d, e, f, g and h each denote an atomic ratio of each element, and when a=12, b=0.01 to 3, c=0 to 8, d=0 to 12, e=0 to 8, f=0.001 to 2, and g=0 to 20, and h is an oxygen atomic ratio required to satisfy a valence of the each component.)
[9]: A method of producing a catalyst comprising at least molybdenum and bismuth, wherein the method comprises the following steps (i) to (v):
(i) mixing at least a molybdenum raw material and a bismuth raw material with a solvent to obtain a slurry (liquid A);
(ii) stirring the liquid A at a temperature of 1 to 30° C. lower than the boiling point of the solvent for 20 to 90 minutes to obtain a slurry (liquid B);
(iii) stirring the liquid B at a temperature of 2° C. or higher than the temperature in the step (ii) for 10 minutes to 10 hours to obtain a slurry (liquid C);
(iv) drying the liquid C to obtain a dried product; and
(v) calcining the dried product to obtain a catalyst.
   [10]: The method of producing the catalyst according to [9], wherein 50% by mass or more of the total solvent is water in the step (i).
   [11]: The method of producing the catalyst according to [9] or [10], wherein the temperature in the step (iii) is 1 to 20° C. higher than the boiling point of the solvent.
   [12]: The method of producing a catalyst according to any one of [9] to [11], wherein the liquid B is stirred for 90 minutes to 10 hours to obtain the liquid C in the step (iii).
   [13]: The method of producing a catalyst according to any one of [9] to [12], comprising producing a catalyst for use in the production an α,β-unsaturated aldehyde and/or an α,β-unsaturated carboxylic acid from an alkene, an alcohol, or an ether.
   [14]: The method of producing a catalyst according to any one of [9] to [13], wherein a catalyst having a composition represented by the following formula (1) is produced:

MoₐBi_{b}Fe_{c}M_{d}XₑY_{f}Si_{g}Oₕ (1)

(wherein in the above formula (1), Mo, Bi, Fe, Si and O each represent molybdenum, bismuth, iron, silicon and oxygen, respectively; M represents at least one element selected from the group consisting of cobalt and nickel; X represents at least one element selected from the group consisting of zinc, chromium, lead, manganese, calcium, magnesium, niobium, silver, barium, tin, tantalum, tungsten, antimony, phosphorus, boron, sulfur, selenium, tellurium, cerium and titanium; Y represents at least one element selected from the group consisting of cesium, lithium, sodium, potassium, rubidium, and thallium; a, b, c, d, e, f, g and h each represents an atomic ratio of each element, and when a=12, b=0.01 to 3, c=0 to 8, d=0 to 12, e=0 to 8, f=0.001 to 2, and g=0 to 20, and h is an oxygen atomic ratio required to satisfy a valence of the each component.)
[15]: A method of producing an α,β-unsaturated aldehyde and/or an α,β-unsaturated carboxylic acid, comprising producing the α,β-unsaturated aldehyde and/or the α,β-unsaturated carboxylic acid from an alkene, an alcohol or an ether by using the catalyst according to any one of [1] to [8].
[16]: A method of producing an α,β-unsaturated aldehyde and/or an α,β-unsaturated carboxylic acid, comprising producing the α,β-unsaturated aldehyde and/or the α,β-unsaturated carboxylic acid from an alkene, an alcohol or an ether by using a catalyst produced by the production method according to any one of [9] to [14].
[17]: A method of producing an α,β-unsaturated carboxylic acid, comprising producing the α,β-unsaturated carboxylic acid from an α,β-unsaturated aldehyde produced by the production method according to [15] or [16].
[18]: A method of producing an α,β-unsaturated carboxylic acid ester, comprising producing the α,β-unsaturated carboxylic acid ester from an α,β-unsaturated carboxylic acid produced by the production method according to any one of [15] to [17].

### Effects of the Invention

According to the present invention, a catalyst with high selectivity of target products can be provided.

### MODE FOR CARRYING OUT THE INVENTION

Embodiments according to the present invention will be described below, however, the present invention is not limited as described below. Moreover, the phrase “XX or more and YY or less” or “XX to YY,” which denotes a numerical value range, refers to a numerical value range including lower limit and upper limit, which are the endpoints, unless otherwise specified. When numerical ranges are described stepwisely, the upper limit and lower limit of each numerical range can be arbitrarily combined.

### Catalyst

The catalyst according to the present invention is a catalyst containing at least molybdenum and bismuth, and when a ratio of the amount of bismuth atoms to the amount of molybdenum atoms, calculated from ICP (inductively coupled plasma) atomic emission spectrometry is A, and a ratio of a peak area of bismuth atoms to a peak area of molybdenum atoms, measured by X-ray photoelectron spectroscopy is B, a B/A is 1.3 to 5. Using such a catalyst allows a target product to be produced from a raw material with high selectivity.

The catalyst according to the present invention is preferably an oxidation catalyst from the viewpoint of selectivity of a target product, and more preferably a catalyst used when an α,β-unsaturated aldehyde and/or an α,β-unsaturated carboxylic acid are/is produced. Specifically, it is preferably a catalyst for producing an α,β-unsaturated aldehyde and/or an α,β-unsaturated carboxylic acid from an alkene, alcohol or ether. Incidentally, the phrase “producing an α,β-unsaturated aldehyde and/or an α,β-unsaturated carboxylic acid” denotes that one of α,β-unsaturated aldehyde and α,β-unsaturated carboxylic acid may be produced or both thereof may be produced.

### Composition of Catalyst

The catalyst according to the present invention contains at least molybdenum and bismuth, and preferably has a composition represented by the following formula (1). Note, however, catalyst components may contain a small amount of elements not listed in the following formula (1).

MoₐBi_{b}Fe_{c}M_{d}XₑY_{f}Si_{g}Oₕ (1)

wherein in the above formula (1), Mo, Bi, Fe, Si and O each represent molybdenum, bismuth, iron, silicon and oxygen, respectively; M represents at least one element selected from the group consisting of cobalt and nickel; X represents at least one element selected from the group consisting of zinc, chromium, lead, manganese, calcium, magnesium, niobium, silver, barium, tin, tantalum, tungsten, antimony, phosphorus, boron, sulfur, selenium, tellurium, cerium and titanium; Y represents at least one element selected from the group consisting of cesium, lithium, sodium, potassium, rubidium, and thallium; a, b, c, d, e, f, g and h each denote an atomic ratio of each element, and when a=12, b=0.01 to 3, c=0 to 8, d=0 to 12, e=0 to 8, f=0.001 to 2, and g=0 to 20, and h is an oxygen atomic ratio required to satisfy a valence of the each component.

In formula (1) above, from the viewpoint of improving the selectivity of a target product, when a=12, the lower limit of b is preferably 0.03 or more, and more preferably 0.05 or more. The upper limit of b is also preferably 2 or less and more preferably 1 or less. The lower limit of c is preferably 0.01 or more, more preferably 0.1 or more, further preferably 1 or more, and particularly preferably 3 or more. The upper limit of c is also preferably 6 or less and more preferably 4 or less.

The catalyst contains molybdenum, bismuth, and, if necessary, iron and may also contain as other elements excluding these elements, the M element, the X element, and the Y element in formula (1) above. Among the other elements, the M element is preferably contained, and the Y element is also preferably contained.

In formula (1) above, from the viewpoint of improving the selectivity of a target product, when a=12, the lower limit of d is preferably 0.01 or more, more preferably 0.1 or more, further preferably 1 or more, and particularly preferably 3 or more. The upper limit of d is also preferably 10 or less and more preferably 9 or less. The lower limit of e is preferably 0.1 or more, more preferably 0.2 or more, and further preferably 0.5 or more. The upper limit of e is also preferably 6 or less and more preferably 4 or less. The lower limit of f is preferably 0.05 or more, more preferably 0.1 or more, and further preferably 0.2 or more. The upper limit of f is also preferably 1.8 or less, more preferably 1.6 or less, and further preferably 1.4 or less.

The catalyst may also have a support for supporting the above elements. The supports are not particularly limited, and include silica, alumina, silica-alumina, magnesia, titania, silicon carbide, and the like. Of these, silica is preferred as a support in order to prevent the support itself from reacting when a support is used. Note, however, when the support is used for a catalyst in the present invention, catalysts including the support are regarded as the catalyst.

In formula (1) above, from the viewpoint of improving the selectivity of the target product, when a=12, the upper limit of g is preferably 20 or less, more preferably 15 or less, and further preferably 10 or less.

Incidentally, a composition of the catalyst is determined by analyzing components of the catalyst dissolved in ammonia water by ICP atomic emission spectrometry. For example, an ICP Optima 8300 (manufactured by Perkin Elmer Corp.) can be used as an analysis apparatus. The analysis conditions are as follows: power: 1300 W, plasma gas flow: 10 L/min, auxiliary gas flow: 0.2 L/min, nebulizer gas flow: 0.55 L/min, detector: split-array type CCD. The ICP atomic emission spectrometry is a method of measuring spectral lines that are emitted when plasma energy is externally applied to a sample, atoms contained therein are excited, and then the excited atoms return to a lower energy level.

### Bismuth Composition on Catalyst Surface with Respect to That of Entire Catalyst

When a ratio of the amount of bismuth atoms to the amount of molybdenum atoms, calculated from ICP atomic emission spectrometry is A, and a ratio of a peak area of bismuth atoms to a peak area of molybdenum atoms, measured by X-ray photoelectron spectroscopy is B, a B/A of the catalyst according to the present invention is 1.3 to 5. Here, the A represents a ratio of the amount of bismuth atoms to the amount of molybdenum atoms in the entire catalyst, and the B represents a ratio of the amount of bismuth atoms to the amount of molybdenum atoms on a catalyst surface. In other words, the B/A represents the amount of bismuth atoms on the catalyst surface with respect to the amount of bismuth atoms in the entire catalyst.

Satisfying the B/A of the catalyst within the aforementioned range enables production of a target product from raw materials thereof with high selectivity. The reason therefore is not clear, however, is considered to be as follows: Bismuth plays a role as an active site of reaction on a catalyst surface, and the B/A being 1.3 or more, i.e., the amount of bismuth atoms on the catalyst surface being sufficient, thereby allows a selective oxidation reaction to a target product to proceed and improves selectivity of the target product. Moreover, the B/A of 5 or less, i.e., no excessive amount of bismuth atoms on the catalyst surface, is considered to inhibit sequential reactions from the target product and inhibit the selectivity of the target product from being reduced.

Note, however, among these described above, the lower limit of the B/A value is preferably 1.5 or higher, more preferably 1.7 or higher, and further preferably 1.9 or higher. The upper limit of the B/A value is also preferably 4 or less and more preferably 3 or less.

The lower limit of the A value is preferably 0.02 or more and more preferably 0.03 or more. The upper limit of the A value is also preferably 0.1 or less and more preferably 0.09 or less.

The lower limit of the B value is preferably 0.04 or more, more preferably 0.06 or more, and further preferably 0.07 or more. The upper limit of the B value is also preferably 0.2 or less, more preferably 0.18 or less, and further preferably 0.16 or less.

Methods of controlling the A value, the B value, the B/A value include a method of adjusting a type and the amount of molybdenum raw material, a type and the amount of bismuth raw material, an agitation time, a heating time, and a heating temperature, and the like in a production method of the catalyst. Among them, the A value, the B value, and the B/A value can be controlled to desired ranges by stirring for 20 to 90 minutes at a temperature of 1 to 30° C. lower than the boiling point of a solvent, in particular in step (ii) described below and stirring for 10 minutes to 10 hours at a temperature of 2° C. or higher than the boiling point of the solvent in step (iii).

In the present invention, the A value is obtained by subjecting the catalyst to ICP atomic emission spectrochemical analysis, as described above and calculating a ratio of the amount of bismuth atoms to the amount of molybdenum atoms. The B value is also obtained by subjecting the catalyst to X-ray photoelectron spectroscopic analysis and calculating a ratio of a peak area of bismuth atoms to a peak area of molybdenum atoms. As an analysis apparatus, for example, a Quantera II (manufactured by ULVAC PHI, INCORPORATED) can be used. The analysis conditions are as follows: X-ray: HP mode-monochromatized Al line source, output: 300 W, acquisition angle: 45°, X-ray beam diameter: 100 μmφ, and linear scanning in the range of 1400 μm. The X-ray photoelectron spectroscopy is a method where X-rays are irradiated onto a sample surface to measure kinetic energy of photoelectrons emitted from the sample surface, from which a composition and chemical state of elements constituting the sample surface can be determined. Since information on elements present within a few nm or less of the sample surface can generally be obtained, information on a composition and chemical state of a surface of a catalyst can be obtained.

### Density of the Catalyst

The density of the catalyst is not particularly limited, but the lower limit is preferably 0.2 g/cm³ or more, more preferably 0.5 g/cm³ or more, and further preferably 1 g/cm³ or more, from the viewpoint of improving durability of the catalyst. The upper limit is, on the other hand, preferably 50 g/cm³ or less, more preferably 30 g/cm³ or less, and further preferably 20 g/cm³ or less, from the viewpoint of improving selectivity of a target product.

### Production Method of Catalyst

Another embodiment of the present invention is a method of producing a catalyst containing at least molybdenum and bismuth and the method includes the following steps (i) to (v). The obtained catalyst preferably has the B/A described above of 1.3 to 5.
(i) A step of mixing at least a molybdenum raw material and a bismuth raw material with a solvent to obtain a slurry (liquid A).
(ii) A step of stirring the liquid A at a temperature of 1 to 30° C. lower than the boiling point of the solvent for 20 to 90 minutes to obtain a slurry (liquid B).
(iii) A step of stirring the liquid B at a temperature of 2° C. or higher than the temperature in step (ii) above for 10 minutes to 10 hours to obtain a slurry (liquid C).
(iv) A step of drying the liquid C to obtain a dried product.
(v) A step of calcining the dried product to obtain a catalyst.

Moreover, the method of producing the catalyst according to the present invention may further employ a forming step, which will be described below.

Each step will be described in detail below.

### Step (i)

In step (i), at least a molybdenum raw material and a bismuth raw material are mixed with a solvent to obtain a slurry (liquid A). The liquid A is prepared by mixing raw materials of molybdenum and bismuth with a solvent. Moreover, raw materials for each element contained in formula (1) described above (hereinafter also referred to as catalyst raw materials) may be further mixed. The amounts of catalyst raw materials used are appropriately adjusted so that a desired catalyst composition is achieved.

The catalyst raw materials are not particularly limited, and each element of nitrates, carbonates, bicarbonates, acetates, ammonium salts, sulfates, oxides, chlorides, hydroxides, halides, oxoacids, oxoacid salts, and the like may be used singly, or in combinations of two or more types thereof.

Examples of the molybdenum raw material include ammonium paramolybdate, molybdenum trioxide, molybdic acid, molybdenum chloride, and the like, and the ammonium paramolybdate is preferably used. Examples of the bismuth raw material include bismuth nitrate, bismuth oxide, bismuth subcarbonate, and the like, and the bismuth oxide is preferably used. Examples of the iron raw material include iron nitrate, iron hydroxide, iron trioxide, and the like, with the iron nitrate being preferably used.

A solvent is not particularly limited as long as it can dissolve or disperse catalyst raw materials, but it preferably contains at least water, and more preferably it contains water in an amount of 50% by mass or more of the total solvent, and further preferably it contains water in an amount of 80% by mass or more of the total solvent, and water may be used singly. The solvent may also contain organic solvents. Examples of organic solvents include but not particularly limited thereto, alcohols, acetone, and the like. The amount of solvent used is not particularly limited, but is preferably 30 to 400 parts by mass relative to 100 parts by mass of the total catalyst raw materials.

Step (i) preferably includes the following steps (i-1) and (i-2).
(i-1) A step of preparing a solution or slurry (liquid A1) containing molybdenum, bismuth, and the X and Y elements in formula (1) above, and a solution or slurry (liquid A2) containing iron and the M element in formula (1) above.
(ii-2) A step of mixing the liquid A1 and liquid A2 to prepare liquid A.

Each step will be described in detail below.

### Step (i-1)

In step (i-1), a solution or slurry (liquid A1) containing molybdenum, bismuth, and the X and Y elements in formula (1) above, and a solution or slurry (liquid A2) containing iron and the M element in formula (1) above, are prepared. Incidentally, the order of preparation of the liquid A1 and liquid A2 is not limited, and the liquid A1 and liquid A2 may be prepared simultaneously.

The amount of each catalyst raw material used is preferably adjusted so that the resulting catalyst has the composition represented by formula (1) above.

The amount of solvent used is not particularly limited, but that of liquid A1 is preferably 70 to 400 parts by mass relative to 100 parts by mass of the total catalyst raw materials. The amount of liquid A2 is preferably 30 to 230 parts by mass relative to 100 parts by mass of the catalyst raw materials.

### Step (i-2)

In step (i-2), the liquid A1 and liquid A2 obtained in step (i-1) above are mixed to prepare liquid A.

### Step (ii)

In step (ii), the liquid A obtained in step (i) is stirred for 20 to 90 minutes at a temperature of 1 to 30° C. lower than the boiling point of the solvent to obtain a slurry (liquid B). For example, in the case of having used water as the solvent in step (i) above, the liquid A is stirred at 70 to 99° C. in step (ii) because the boiling point of water is 100° C. Note, however, in a case in which a plurality of solvents with different boiling points is used in step (i), they are stirred at a temperature of 1 to 30° C. lower than a boiling point of a solvent with the largest mass fraction.

In step (ii), when catalyst raw materials are dissolved in a solvent, solubility of the bismuth raw material is adjusted to a constant level by setting a temperature and stirring time to the conditions thereof described above. Therefore, it is conjectured that when a bismuth-molybdate mixed oxide layer is formed in step (iii) described below, bismuth, which serves as an active point, suitably precipitates on its surface, and a catalyst with a B/A of 1.5 to 5 can be obtained. When the temperature in step (ii) is lower than specified or the stirring time is shorter than specified, the solubility of the bismuth raw material becomes low, which thereby results in the B/A of the obtained catalyst less than 1.5. In the case of the temperature in step (ii) being higher than specified or the stirring time being longer than specified, the solubility of the molybdenum raw material and bismuth raw material increases, which thereby tends to increase the B/A of the resulting catalyst to greater than 5.

The upper limit of a temperature when stirring the liquid A is preferably 3° C. or higher below the boiling point of the solvent and more preferably 5° C. or higher below the boiling point of the solvent. The lower limit is also preferably a temperature 25° C. or lower below the boiling point of the solvent, more preferably 20° C. or lower, and further preferably or lower.

The lower limit of a time for stirring at the aforementioned temperature range is preferably 30 minutes or longer and more preferably 40 minutes or longer, and the upper limit thereof is preferably 80 minutes or shorter, and more preferably 70 minutes or shorter.

### Step (iii)

In step (iii), the liquid B obtained in step (ii) above is stirred for 10 minutes to 10 hours at a temperature of 2° C. or higher than the temperature in step (ii) to obtain a slurry (liquid C).

In step (iii), a bismuth-molybdate mixed oxide layer is formed. In this case, it is considered that stirring the liquid B, in which solubility of bismuth was adjusted in step (ii) above, at the temperature and for the time, described above, allows bismuth that is to be an active site when forming the bismuth-molybdate mixed oxide layer to suitably precipitate on the surface to obtain a catalyst with a B/A of 1.5 to 5. The temperature in step (iii) being lower than specified or a stirring time being shorter than specified does not promote precipitation of bismuth on its surface, resulting in that the B/A of the obtained catalyst tends to be less than 1.5. The temperature in step (iii) being higher than specified or the stirring time being longer than specified, resulting in excessive precipitation of bismuth on its surface, and therefore the B/A of the resulting catalyst tends to be greater than 5.

The lower limit of a temperature when stirring the liquid B is preferably 3° C. or higher above the temperature in step (ii), more preferably 5° C. or higher, further preferably 6° C. or higher, and particularly preferably 8° C. or higher. The upper limit is preferably 20° C. or lower above the temperature of step (ii), and more preferably 10° C. or lower.

Moreover, a temperature at which the liquid B is stirred is preferably a temperature of 1 to 20° C. higher than the boiling point of the solvent. For example, when water is used as the solvent in step (i), the liquid B is preferably stirred at 101 to 120° C. in step (iii) because the boiling point of water is 100° C. The lower limit of a temperature at which the liquid B is stirred is more preferably 2° C. or higher above the boiling point of the solvent, and more preferably 3° C. or higher. The upper limit is also more preferably 10° C. or lower above the boiling point of the solvent, and further preferably 5° C. or lower.

The lower limit of time for stirring in the temperature range described above is preferably 20 minutes or longer, more preferably 30 minutes or longer, and further preferably 60 minutes or longer, particularly preferably 90 minutes or longer, and most preferably 2 hours or longer. The upper limit is also preferably 9 hours or shorter and more preferably 8 hours or shorter.

### Step (iv)

In step (iv), the liquid C obtained in step (iii) above is dried to obtain a dried product.

For drying the liquid C, publicly known methods such as a drum drying method, an air flow drying method, an evaporation solidification method, and a spray drying method, can be used. A drying temperature is preferably 120 to 500° C., with the lower limit of 140° C. or higher and the upper limit of 350° C. or lower being more preferred. Drying is preferably carried out so that the moisture content of the resulting dried product is 0.1 to 4.5% by mass. Note, however, these conditions can be appropriately selected depending on a shape and size of a desired catalyst. Implementing drying of liquid C can inhibit a dried product from adhering and improve yield.

### Step (v)

In step (v), the dried product obtained in step (iv) above is calcined to obtain a catalyst. The calcination can be carried out after the forming step described below is performed to obtain a formed product, however, it is preferably carried out before the forming step from the viewpoint of a catalyst strength. In the present invention, catalysts including those after calcination and after forming are collectively referred to as catalysts.

The calcination may be carried out only once, or it may be divided into a plurality of times together with the forming step described below. For example, primary calcination may be carried out first, the forming step described below may be carried out for the resulting primarily calcinated product, and then secondary calcination may be carried out for the resulting formed product. Moreover, the primary calcination and secondary calcination may be carried out, and the forming step may be carried out for the resulting catalyst.

The calcination is preferably carried out under an oxygen-containing gas (for example air) distribution, or under inert gas distribution. The term “inert gas” refers to a gas that does not lower catalytic activity, such as nitrogen, carbon dioxide, helium, and argon.

A calcination temperature is preferably 200 to 700° C. The lower limit of the calcination temperature is more preferably 300° C. or higher, while the upper limit is more preferably 500° C. or lower and further preferably 450° C. or lower.

A calcination time is preferably 0.5 to 40 hours, while the lower limit is more preferably 1 hour or longer. It is noted that the calcination time refers to a time required to hold a predetermined calcination temperature after it was reached.

Of these described above, it is preferred that a dried product underwent primary calcination, followed by forming, and then the resulting formed product undergoes secondary calcination.

In this case, a calcination temperature of the primary calcination is preferably 200 to 600° C., with the lower limit of 250° C. or higher and the upper limit of 450° C. or lower being more preferred. A calcination time of the primary calcination is preferably 0.5 to 5 hours from the viewpoint of improving selectivity of a target product. A type of calcination furnace and calcination methods upon the primary calcination are not particularly limited, and for example, a box-type calcination furnace, a tunnel furnace type calcination furnace or the like may be used to calcinate a dried product or a formed product in a fixed condition. Moreover, a rotary kiln and the like may be used to calcinate the dried product or formed product while it is flowed.

A calcination temperature of the secondary calcination is preferably 300 to 700° C., with the lower limit of 400° C. or higher and the upper limit of 600° C. or lower being more preferred. A calcination time of the secondary calcination is preferably 10 minutes to 10 hours from the viewpoint of improving selectivity of a target product, and the lower limit thereof is more preferably 1 hour or longer. A type of calcination furnace and calcination methods upon the secondary calcination are not particularly limited, and for example, a box-type calcination furnace, a tunnel furnace type calcination furnace or the like may be used to calcinate a formed product or a primarily calcinated product in a fixed condition. Moreover, a rotary kiln and the like may be used to calcinate the dried product or primarily calcinated product while it is flowed.

### Forming Step

In the forming step, the dried product obtained in step (iv) above or the calcinated product obtained in step (v) above is formed to obtain a formed product. The forming method is not particularly limited, and any publicly known dry or wet forming method can be applied. Examples thereof include tableting forming, extrusion forming, pressure forming, rolling granulation, and the like.

Upon forming, publicly known additives, for example, organic compounds such as a polyvinyl alcohol and a carboxymethylcellulose, may be added. Furthermore, inorganic compounds such as graphite and silicon soil, and inorganic fibers such as glass fibers, ceramic fibers, and carbon fibers may be added.

A shape of the formed product is not particularly limited and can be arbitrary shapes, such as spherical, cylindrical, ring, star shape, or a granular shape of a formed product that was crushed and classified after forming, and the like. Of these, preferable are spherical, cylindrical, and ring shapes from the viewpoint of a mechanical strength. A size of the formed product is not particularly limited, but it is preferably, 0.1 to 10 mm of a diameter of sphere, for example, in the case of a spherical shape. The lower limit of the diameter of the sphere is more preferably 0.5 mm or larger, further preferably 1 mm or larger, and particularly preferably 3 mm or larger. The upper limit of the diameter of the sphere is also more preferably 8 mm or smaller and further preferably 6 mm or smaller. In the case of a ring shape or cylinder shape, a diameter and height of a circle at the bottom of the ring or cylinder are both preferably 0.1 to 10 mm. The lower limit of the diameter and height are more preferably 0.5 mm or larger, further preferably 1 mm or larger, and particularly preferably 3 mm or larger. The upper limit of the diameter and height are also more preferably 8 mm or smaller and further preferably 6 mm or smaller. For other shapes, a length between the two most distant points in a solid body of a catalyst is preferably 0.1 to 10 mm. The lower limit of the length between two points is more preferably 0.5 mm or more, further preferably 1 mm or more, and particularly preferably 3 mm or more. The upper limit of the length between the two points is also more preferably 8 mm or less and further preferably 6 mm or less. This improves the selectivity of a target product and a catalyst life.

An outer surface area of a formed product is not particularly limited, but from the viewpoint of stable production of a target product over a long period of time, the lower limit thereof is preferably 0.01 cm² or more, more preferably 0.05 cm² or more, and further preferably 0.1 cm² or more. From the viewpoint of improving selectivity of a target product, the upper limit is, on the other hand, preferably 4 cm² or less, more preferably 3 cm² or less, and further preferably 2 cm² or less.

The volume of a formed product is not particularly limited, but from the viewpoint of stable production of a target product over a long period of time, the lower limit thereof is preferably 0.0002 cm³ or more, more preferably 0.002 cm³ or more, and further preferably 0.02 cm³ or more. From the viewpoint of improving selectivity of a target product, the upper limit is, on the other hand, preferably 5 cm³ or less, more preferably 1 cm³ or less, and further preferably 0.5 cm³ or less.

The mass of a formed product is not particularly limited, but from the viewpoint of stable production of a target product over a long period of time, the lower limit thereof is preferably 0.002 g/product or more, more preferably 0.01 g/product or more, and further preferably 0.05 g/product or more. From the viewpoint of improving selectivity of a target product, the upper limit is, on the other hand, preferably 0.5 g/product or less, more preferably 0.3 g/product or less, and further preferably 0.2 g/product or less.

The filling bulk density of a formed product is not particularly limited, but from the viewpoint of stable production of a target product over a long period of time, the lower limit thereof is preferably 0.2 g/cm³ or higher, more preferably 0.3 g/cm³ or higher, and further preferably 0.4 g/cm³ or higher. From the viewpoint of improving selectivity of a target product, the upper limit is, on the other hand, preferably 1 g/cm³ or lower, more preferably 0.9 g/cm³ or lower, and further preferably 0.8 g/cm³ or lower. Incidentally, the filling bulk density of the formed product shall refer to a value calculated from the total mass of a formed product when filled into a 100 ml graduated cylinder by the method in accordance with JIS-K 7365.

The resulting formed product may be supported on a support. Examples of supports used upon supporting include silica, alumina, silica-alumina, magnesia, titania, silicon carbide, and the like. The formed product can also be diluted with inert substances such as silica, alumina, silica-alumina, magnesia, titania, and silicon carbide and used.

The catalyst can be produced as described above.

### Method of Producing α,β-Unsaturated Aldehyde and/or α,β-Unsaturated Carboxylic Acid

In the method of producing an α,β-unsaturated aldehyde and/or an α,β-unsaturated carboxylic acid, the catalyst according to the present invention or a catalyst produced by the production method according to the present invention is used to produce from an alkene, alcohol or ether, the corresponding α,β-unsaturated aldehyde and/or α,β-unsaturated carboxylic acid.

Examples of the alkenes include propylene, isobutylene, and the like. Examples of the alcohols also include t-butyl alcohol, isobutyl alcohol, and the like. Examples of the ethers also include methyl-t-butyl ether and the like. Oxidation of these raw organic compounds enables production of the corresponding α,β-unsaturated aldehydes and/or α,β-unsaturated carboxylic acids. For example, in the case of the raw organic compound being propylene, the corresponding α,β-unsaturated aldehyde is acrolein, and the corresponding α,β-unsaturated carboxylic acid is acrylic acid. Moreover, in a case in which the raw organic compound is isobutylene, t-butyl alcohol, isobutyl alcohol, or methyl-t-butyl ether, the corresponding α,β-unsaturated aldehyde is methacrolein, and the corresponding α,β-unsaturated carboxylic acid is methacrylic acid. From the viewpoint of selectivity of a target product, the α,β-unsaturated aldehyde and α,β-unsaturated carboxylic acid are preferably methacrolein and methacrylic acid, respectively.

The method of producing an α,β-unsaturated aldehyde and/or an α,β-unsaturated carboxylic acid according to the present invention can be carried out by contacting the catalyst according to the present invention or a catalyst produced by the production method according to the present invention, and a raw material gas containing the raw organic compounds and oxygen in a reactor.

The reactor is not particularly limited, but a tube reactor equipped with a reaction tube filled with a catalyst is preferably used, and industrially a multi-tube reactor equipped with a plurality of reaction tubes is more preferably used. A catalyst layer inside the reactor may be a single catalyst layer, or a plurality of catalysts with different activities may be each separated and filled to a plurality of layers. The catalyst may also be diluted with an inert support to control the activity and then filled.

A concentration of a raw organic compound in a raw material gas is preferably 1 to 20% by volume, with the lower limit of 3% by volume or more and the upper limit of 10% by volume or less being more preferred. Note, however, the raw organic compound may contain a small amount of impurities such as lower saturated alkanes that do not substantially affect the present reaction.

A concentration of oxygen in the raw material gas is preferably 0.1 to 5 moles relative to 1 mole of raw organic compound, with the lower limit of 0.5 moles or more and the upper limit of 3 moles or less being more preferred. Air is preferred as an oxygen source for the raw material gas from an economic point of view. Gas enriched with oxygen obtained by mixing pure oxygen with air or the like may also be used, if necessary.

The raw material gas may be diluted with an inert gas such as nitrogen, carbon dioxide gas or the like from an economic standpoint. Furthermore, water vapor may be added to the raw material gas. Reaction in the presence of water vapor enables an α,β-unsaturated aldehyde and/or an α,β-unsaturated carboxylic acid to be obtained at higher selectivity. A concentration of water vapor in the raw material gas is preferably 0.1 to 50% by volume, with the lower limit of 1% by volume or more and the upper limit of 40% by volume or less being more preferred.

A reaction pressure is preferably 0 to 1 MPa (G). Here, “(G)” is a gauge pressure, and 0 MPa (G) means that the reaction pressure is atmospheric pressure. A reaction temperature is also preferably 200 to 450° C. with the lower limit of 250° C. or higher and the upper limit of 400° C. or lower being more preferred.

A contact time between the raw material gas and the catalyst is preferably 0.5 to 15 seconds. The lower limit of the contact time is more preferably 1 second or longer, while the upper limit is more preferably 10 seconds or shorter and further preferably 5 seconds or shorter.

The production in the manner described above allows α,β-unsaturated aldehydes and/or α,β-unsaturated carboxylic acids corresponding to the raw organic compounds used to be obtained with high selectivity.

### Method of Producing α,β-Unsaturated Carboxylic Acid

In the method of producing an α,β-unsaturated carboxylic acid, from an α,β-unsaturated aldehyde produced by the production method according to the present invention, the corresponding α,β-unsaturated carboxylic acid or the like is produced.

Examples of the α,β-unsaturated aldehydes include (meth)acrolein, crotonaldehyde (β-methyl acrolein), cinnamaldehyde (β-phenyl acrolein), and the like. The α,β-unsaturated carboxylic acid to be produced is an α,β-unsaturated carboxylic acid in which an aldehyde group of the α,β-unsaturated aldehyde was changed to a carboxyl group. Specifically, when the α,β-unsaturated aldehyde is (meth)acrolein, (meth)acrylic acid is obtained. From the viewpoint of selectivity of the target product, the α,β-unsaturated aldehyde and the α,β-unsaturated carboxylic acid are preferably (meth)acrolein and (meth)acrylic acid, respectively and more preferably methacrolein and methacrylic acid. Note that “(meth)acrolein” refers to acrolein and methacrolein, and “(meth)acrylic acid” refers to acrylic acid and methacrylic acid.

The method of producing an α,β-unsaturated carboxylic acid according to the present invention can be carried out by contacting the catalyst according to the present invention or a catalyst produced by the production method according to the present invention, and a raw material gas containing an α,β-unsaturated aldehyde and oxygen in a reactor. As the catalyst, a heteropoly acid catalyst or the like is preferably used. As the reactor, a reactor similar to that used in the method of producing an α,β-unsaturated aldehyde and/or an α,β-unsaturated carboxylic acid, described above, can be used. A catalyst layer in the reactor may be a single layer, or a plurality of catalysts with different activities may be each separated and filled to a plurality of layers. The catalyst may also be diluted with an inert support to control the activity and then filled.

A concentration of α,β-unsaturated aldehyde in the raw material gas is preferably 1 to 20% by volume, with the lower limit of 3% by volume or more and the upper limit of 10% by volume or less being more preferred. Note, however, the α,β-unsaturated aldehyde may contain a small amount of impurities such as lower saturated aldehydes that do not substantially affect the present reaction.

A concentration of oxygen in the raw material gas is preferably 0.4 to 4 moles relative to 1 mole of α,β-unsaturated aldehyde, with the lower limit of 0.5 moles or more and the upper limit of 3 moles or less being more preferred. Air is preferred as an oxygen source for the raw material gas from an economic point of view. Gas enriched with oxygen by mixing pure oxygen with air or the like may be also used, if necessary.

From an economic standpoint, the raw material gas may be diluted with an inert gas such as nitrogen or a carbon dioxide gas. Furthermore, water vapor may be added to the raw material gas. A reaction in the presence of water vapor enables an α,β-unsaturated carboxylic acid to be obtained at higher selectivity. A concentration of water vapor in the raw material gas is preferably 0.1 to 50% by volume, with the lower limit of 1% by volume or more and the upper limit of 40% by volume or less being more preferred.

A reaction pressure is preferably 0 to 1 MPa (G). A reaction temperature is also preferably 200 to 450° C., with the lower limit of 250° C. or higher and the upper limit of 400° C. or lower being more preferred.

A contact time between the raw material gas and the catalyst is preferably 0.5 to 15 seconds. The lower limit is more preferably 1 second or longer, while the upper limit is more preferably 10 seconds or shorter and further preferably seconds or shorter.

### Production Method of α,β-Unsaturated Carboxylic Acid Ester

In the method of producing α,β-unsaturated carboxylic acid esters according to the present invention, an α,β-unsaturated carboxylic acid produced by the production method according to the present invention, is esterified. Alcohols to be reacted with α,β-unsaturated carboxylic acids are not particularly limited, and examples thereof include methanol, ethanol, propanol, isopropanol, butanol, isobutanol, and the like. Examples of the resulting α,β-unsaturated carboxylic acid esters include, for example, methyl (meth)acrylate, ethyl (meth) acrylate, propyl (meth)acrylate, isopropyl (meth) acrylate, butyl (meth)acrylate, isobutyl (meth)acrylate, and the like. The reaction can be carried out in the presence of an acidic catalyst such as a sulfonic acid type cation exchange resin. A reaction temperature is preferably 50 to 200° C.

### EXAMPLES

The present invention will be described in detail by way of Examples and Comparative Examples, however, the present invention is not limited to these Examples. Note, however, the “parts” in Examples and Comparative Examples refer to parts by mass.

### Composition of Catalyst

A composition of the entire catalyst was determined by analyzing components of a catalyst dissolved in ammonia water by ICP atomic emission spectrometry. An ICP Optima 8300 (manufactured by Perkin Elmer Inc.) was used as an analyzer, with an output of 1300 W, plasma gas flow rate: 10 L/min, auxiliary gas flow rate: 0.2 L/min, nebulizer gas flow rate: 0.55 L/min, and detector: split array type CCD.

A ratio of the amount of bismuth atoms to the amount of molybdenum atoms in a composition of the entire catalyst is obtained to calculate an A value.

### X-Ray Photoelectron Spectroscopic Analysis of Catalyst

X-ray photoelectron spectroscopic analysis of the catalyst was carried out to obtain a ratio of a peak area of bismuth atoms to a peak area of molybdenum atoms for determination of a B value. A Quantera II (manufactured by ULVAC PHI INCORPORATED) with X-ray: HP mode-monochromatized Al source, output: 300 W, acquisition angle: 45°, and X-ray beam diameter: 100 μmφ, was used as an analyzer, and a range of 1400 μm was linearly scanned.

### Reaction Evaluation

Reactions of the catalysts in Examples and Comparative Examples were evaluated by taking production of methacrolein and methacrylic acid by oxidation of isobutylene as an example. The raw material gas and products in the reaction evaluation were analyzed by using the following gas chromatography.

Analysis of methacrolein: A GC-2014 manufactured by Shimadzu Corporation with column: QUADREX 007-CW 20 m×0.32 mm and film thickness: 3 μm.

Analysis of methacrylic acid: A GC-2014 manufactured by Shimadzu Corporation with column: DB-FFAP manufactured by J&W Corporation, 30 m×0.32 mm and film thickness: 1.00 μm

From the results of gas chromatography, the total selectivity of methacrolein and methacrylic acid formed was determined by the following equation.

Total selectivity of methacrolein and methacrylic acid (%)=(P1+P2)/M1×100

In the above formula, M1 is the number of moles of isobutylene reacted per unit time, P1 is the number of moles of methacrolein produced per unit time, and P2 is the number of moles of methacrylic acid produced per unit time.

### Example 1

Liquid A1 was obtained by mixing 500 parts by mass of ammonium paramolybdate tetrahydrate, 12.3 parts by mass of ammonium para tungstate, 27.6 parts by mass of cesium nitrate, 38.5 parts by mass of bismuth (III) oxide, and 20.6 parts by mass of antimony trioxide with 2,000 parts by mass of pure water at 60° C. as a solvent. Separately from the liquid A1, liquid A2 was obtained by mixing 200.2 parts by mass of iron (III) nitrate nonahydrate and 515.1 parts by mass of cobalt (II) nitrate hexahydrate in 1,000 parts by mass of pure water. The liquid A1 and liquid A2 were then mixed to obtain liquid A.

The obtained liquid A was heated to 95° C. and stirred for 1 hour while maintaining the liquid temperature at 95° C. to obtain liquid B.

The resulting liquid B was heated to 101° C. and stirred for 3 hours while maintaining the liquid temperature at 101° C. to obtain liquid C.

The resulting liquid C was dried in a spray dryer to obtain a dried product. The dried product did not adhere to the wall of the spray dryer and was in favorable dry condition.

The resulting dried product underwent primary calcination at 300° C. for 1 hour under an air atmosphere, and then pulverized. The pulverized product after calcination and drying was pressure-formed and then pulverized to obtain pulverized particles. Thereafter the pulverized particles were classified and passed through a sieve with an aperture of 2.36 mm, and pulverized particles that did not pass through a sieve with an aperture of 0.71 mm, were collected. The collected pulverized particles were then subjected to secondary calcination at 500° C. for 3 hours in an air atmosphere to obtain a catalyst.

A composition of the obtained catalyst, excluding oxygen, was Mo₁₂Bi_{0.7}Fe_{2.1}Co_{7.5}W_{0.2}Sb_{0.6}Cs_{0.6}. ICP atomic emission spectrochemical analysis and X-ray photoelectron spectroscopic analysis were also performed on the catalyst. The calculated values of A, B and B/A are shown in Table 1.

The obtained catalyst was then filled in a stainless steel reaction tube to form a catalyst layer, and an oxidation reaction of isobutylene was carried out under the following conditions. The results are shown in Table 1.
Composition of raw material gas: 5% by volume of isobutylene, 12% by volume of oxygen, 10% by volume of water vapor, and 73% by volume of nitrogen
Reaction temperature: 340° C.
Contact time between raw material gas and catalyst: 2.7 seconds.

### Example 2

Liquid A1 was prepared in the same manner as in Example 1, except that the amount of antimony trioxide was 24.8 parts by mass. Moreover, separately from the liquid A1, liquid A2 was obtained in the same manner as in Example 1. The liquid A1 and liquid A2 were then mixed to obtain liquid A.

The obtained liquid A was heated to 95° C. and stirred for 1 hour while maintaining the liquid temperature at 95° C. to obtain liquid B.

The resulting liquid B was heated to 101° C. and stirred for 5 hours while maintaining the liquid temperature at 101° C. to obtain liquid C.

The resulting liquid C was dried in a spray dryer to obtain a dried product. The dried product did not adhere to the wall of the spray dryer and was in favorable dry condition.

The resulting dried product was subjected to primary calcination, forming and secondary calcination in the same manner as in Example 1 to obtain a catalyst.

A composition of the obtained catalyst, excluding oxygen, was Mo₁₂Bi_{0.7}Fe_{2.1}Co_{7.5}W_{0.2}Sb_{0.72}Cs_{0.6}. ICP atomic emission spectrochemical analysis and X-ray photoelectron spectroscopic analysis were also performed on the catalyst. The calculated values of A, B and B/A are shown in Table 1.

The obtained catalyst was then used to evaluate a reaction in the same manner as in Example 1. The results are shown in Table 1.

### Example 3

Liquid A1 was prepared in the same manner as in Example 1, except that the amount of antimony trioxide was 15.5 parts by mass. Moreover, separately from the liquid A1, liquid A2 was obtained in the same manner as in Example 1. The liquid A1 and liquid A2 were then mixed to obtain liquid A.

The obtained liquid A was heated to 95° C. and stirred for 1 hour while maintaining the liquid temperature at 95° C. to obtain liquid B.

The resulting liquid B was heated to 101° C. and stirred for 7 hours while maintaining the liquid temperature at 101° C. to obtain liquid C.

The resulting liquid C was dried in a spray dryer to obtain a dried product. The dried product was not adhered to the wall of the spray dryer and was in favorable dry condition.

The resulting dried product was subjected to primary calcination, forming, and secondary calcination in the same manner as in Example 1 to obtain a catalyst.

A composition of the obtained catalyst, excluding oxygen, was Mo₁₂Bi_{0.7}Fe_{2.1}Co_{7.5}W_{0.2}Sb_{0.45}Cs_{0.6}. ICP atomic emission spectrochemical analysis and X-ray photoelectron spectroscopic analysis were also performed on the catalyst. The calculated values of A, B and B/A are shown in Table 1.

The obtained catalyst was then used to evaluate a reaction in the same manner as in Example 1. The results are shown in Table 1.

### Comparative Example 1

Liquid B was obtained by the same method as in Example 1.

The obtained liquid B was dried in a spray dryer to obtain a dried product. In other words, the dried product was obtained by drying the liquid B without implementing step (iii). The dried product did not adhere to the wall of the spray dryer, indicating that the dried product was in favorable condition.

The dried product was subjected to primary calcination, forming, and secondary calcination in the same manner as in Example 1 to obtain a catalyst.

A composition of the obtained catalyst, excluding oxygen, was Mo₁₂Bi_{0.7}Fe_{2.1}Co_{7.5}W_{0.2}Sb_{0.6}Cs_{0.6}. ICP atomic emission spectrochemical analysis and X-ray photoelectron spectroscopic analysis were also performed on the catalyst. The calculated values of A, B and B/A are shown in Table 1.

The obtained catalyst was then used to evaluate a reaction in the same manner as in Example 1. The results are shown in Table 1.

### Comparative Example 2

Liquid A was obtained by the same method as in Example 1.

The resulting liquid A was heated to 95° C. and stirred for 2 hours while maintaining the liquid temperature at 95° C. In other words, liquid B′ was obtained by stirring for a time longer than 90 minutes in step (ii).

The resulting liquid B′ was heated to 100° C. and stirred for 1 hour while maintaining the liquid temperature at 100° C. to obtain liquid C.

The resulting liquid C was dried in a spray dryer to obtain a dried product. The dried product did not adhere to the wall of the spray dryer, indicating that the dried product was in favorable condition.

The resulting dried product was subjected to primary calcination, forming, and secondary calcination in the same manner as in Example 1 to obtain a catalyst.

A composition of the obtained catalyst, excluding oxygen, was Mo₁₂Bi_{0.7}Fe_{2.1}Co_{7.5}W_{0.2}Sb_{0.6}Cs_{0.6}. ICP atomic emission spectrochemical analysis and X-ray photoelectron spectroscopic analysis were also performed on the catalyst. The calculated values of A, B and B/A are shown in Table 1.

The obtained catalyst was then used to evaluate a reaction in the same manner as in Example 1. The results are shown in Table 1.

TABLE 1

| | | | | | | | | Total |
|---|---|---|---|---|---|---|---|---|
| | | | | | | | | select- |
| | | | | | | | | ivity of |
| | | | | | | | | metha- |
| | Step (ii) | | Step (iii) | | | | | crolein |
| | Tem- | | Tem- | | | | | and |
| | per- | | per- | | | | | metha- |
| | ature | Time | ature | Time | | | B/ | crylic |
| | (° C.) | (hr) | (° C.) | (hr) | A | B | A | acid (%) |
| Example 1 | 95 | 1 | 103 | 3 | 0.05 | 0.10 | 2.0 | 91.2 |
| Example 2 | 95 | 1 | 103 | 5 | 0.05 | 0.11 | 2.3 | 91.2 |
| Example 3 | 95 | 1 | 103 | 7 | 0.05 | 0.08 | 1.6 | 91.0 |
| Comparative | 95 | 1 | — | — | 0.05 | 0.06 | 1.2 | 89.1 |
| Example 1 | | | | | | | | |
| Comparative | 95 | 2 | 100 | 1 | 0.06 | 0.06 | 1.1 | 89.5 |
| Example 2 | | | | | | | | |

As shown in Table 1, Examples 1 to 3 in which each catalyst with the B/A within the specified range was used, exhibited the favorable total selectivity of methacrolein and methacrylic acid.

Incidentally, methacrylic acid can be obtained by oxidizing the methacrolein obtained in Examples, and methacrylic acid ester can be obtained by esterifying the methacrylic acid.

### INDUSTRIAL APPLICABILITY

According to the present invention, a catalyst capable of producing target products such as an α,β-unsaturated aldehyde and/or an α,β-unsaturated carboxylic acid at high selectivity, can be provided, which is industrially useful.

What is claimed is:

1. A catalyst comprising at least molybdenum and bismuth,
wherein
when a ratio of the amount of bismuth atoms to the amount of molybdenum atoms, calculated from ICP (inductively coupled plasma) atomic emission spectrometry is A, and a ratio of a peak area of bismuth atoms to a peak area of molybdenum atoms, measured by X-ray photoelectron spectroscopy is B, a B/A is 1.3 to 5.

2. The catalyst according to claim 1, wherein the B/A value is 1.5 to 4.

3. The catalyst according to claim 1, wherein the B/A value is 1.7 to 3.

4. The catalyst according to claim 1, wherein the A value is 0.02 to 0.1.

5. The catalyst according to claim 1, wherein the B value is 0.04 to 0.2.

6. The catalyst according to claim 1, wherein the B value is 0.07 to 0.16.

7. The catalyst according to claim 1, for use in production of an α,β-unsaturated aldehyde and/or an α,β-unsaturated carboxylic acid from an alkene, an alcohol, or an ether.

8. The catalyst according to claim 1, wherein a catalyst composition is represented by the following formula (1):
MoₐBi_{b}Fe_{c}M_{d}XₑY_{f}Si_{g}Oₕ (1)
(wherein in the above formula (1), Mo, Bi, Fe, Si and O each represent molybdenum, bismuth, iron, silicon and oxygen, respectively; M represents at least one element selected from the group consisting of cobalt and nickel; X represents at least one element selected from the group consisting of zinc, chromium, lead, manganese, calcium, magnesium, niobium, silver, barium, tin, tantalum, tungsten, antimony, phosphorus, boron, sulfur, selenium, tellurium, cerium and titanium; Y represents at least one element selected from the group consisting of cesium, lithium, sodium, potassium, rubidium, and thallium; a, b, c, d, e, f, g and h each denote an atomic ratio of each element, and when a=12, b=0.01 to 3, c=0 to 8, d=0 to 12, e=0 to 8, f=0.001 to 2, and g=0 to 20, and h is an oxygen atomic ratio required to satisfy a valence of the each component.)

9. A method of producing a catalyst comprising at least molybdenum and bismuth, wherein the method comprises the following steps (i) to (v):
(i) mixing at least a molybdenum raw material and a bismuth raw material with a solvent to obtain a slurry (liquid A);
(ii) stirring the liquid A at a temperature of 1 to 30° C. lower than the boiling point of the solvent for 20 to 90 minutes to obtain a slurry (liquid B);
(iii) stirring the liquid B at a temperature of 2° C. or higher than the temperature in the step (ii) for 10 minutes to 10 hours to obtain a slurry (liquid C);
(iv) drying the liquid C to obtain a dried product; and
(v) calcining the dried product to obtain a catalyst.

10. The method of producing the catalyst according to claim 9, wherein 50% by mass or more of the total solvent is water in the step (i).

11. The method of producing the catalyst according to claim 9, wherein the temperature in the step (iii) is 1 to 20° C. higher than the boiling point of the solvent.

12. The method of producing a catalyst according to claim 9, wherein the liquid B is stirred for 90 minutes to 10 hours to obtain the liquid C in the step (iii).

13. The method of producing a catalyst according to claim 9, comprising producing a catalyst for use in the production of an α,β-unsaturated aldehyde and/or an α,β-unsaturated carboxylic acid from an alkene, an alcohol, or an ether.

14. The method of producing a catalyst according to claim 9, comprising producing a catalyst having a composition represented by the following formula (1):
MoₐBi_{b}Fe_{c}M_{d}XₑY_{f}Si_{g}Oₕ (1)
(wherein in the above formula (1), Mo, Bi, Fe, Si and O each represent molybdenum, bismuth, iron, silicon and oxygen, respectively; M represents at least one element selected from the group consisting of cobalt and nickel; X represents at least one element selected from the group consisting of zinc, chromium, lead, manganese, calcium, magnesium, niobium, silver, barium, tin, tantalum, tungsten, antimony, phosphorus, boron, sulfur, selenium, tellurium, cerium and titanium; Y represents at least one element selected from the group consisting of cesium, lithium, sodium, potassium, rubidium, and thallium; a, b, c, d, e, f, g and h each represents an atomic ratio of each element, and when a=12, b=0.01 to 3, c=0 to 8, d=0 to 12, e=0 to 8, f=0.001 to 2, and g=0 to 20, and h is an oxygen atomic ratio required to satisfy a valence of the each component.)

15. A method of producing an α,β-unsaturated aldehyde and/or an α,β-unsaturated carboxylic acid, comprising producing the α,β-unsaturated aldehyde and/or the α,β-unsaturated carboxylic acid from an alkene, an alcohol or an ether by using the catalyst according to claim 1.

16. A method of producing an α,β-unsaturated aldehyde and/or an α,β-unsaturated carboxylic acid, comprising producing the α,β-unsaturated aldehyde and/or the α,β-unsaturated carboxylic acid from an alkene, an alcohol or an ether by using a catalyst produced by the production method according to claim 9.

17. A method of producing an α,β-unsaturated carboxylic acid, comprising producing the α,β-unsaturated carboxylic acid from an α,β-unsaturated aldehyde produced by the production method according to claim 15.

18. A method of producing an α,β-unsaturated carboxylic acid ester, comprising producing the α,β-unsaturated carboxylic acid ester from an α,β-unsaturated carboxylic acid produced by the production method according to claim 15.
